(19) United States
(12) Patent Application Publication
(10) Pub. No.: US 2025/0336521 A1
(43) Pub. Date: Oct. 30, 2025

(54) CELL MANUFACTURING MANAGEMENT PLATFORM USING MACHINE LEARNING

(71) Applicant: Janssen Research & Development, LLC, Raritan, NJ (US)

(72) Inventors: Amar P S Chahal, Fremont, CA (US); Gurparkash Singh, Milton, MA (US); Bart Vannieuwenhuyse, Brecht (BE)

(21) Appl. No.: 18/644,896

(22) Filed: Apr. 24, 2024

(51) Int. Cl. G16H 50/20

(52) U.S. Cl. CPC G16H 50/20

(57) ABSTRACT

A cell manufacturing management platform facilitates management of a cell manufacturing process. The cell manufacturing management platform tracks events associated with a cell manufacturing process and coordinates between disparate entities involved in the process. The cell manufacturing management platform utilizes machine learning techniques to generate inferences associated with event scheduling in a manner that optimizes an efficiency metric and reduces likelihood of exceptions occurring. Machine learning models may furthermore be used to generate various alerts or other actions associated with the process. A user interface enables different participating entities to track progress of the process and upcoming events.

### BACKGROUND

### Technical Field

The described embodiments relate to a cell manufacturing management platform that utilizes machine learning for management and scheduling of interconnected events involving multiple disparate entities.

### Description of the Related Art

Cell manufacturing processes are employed in various medical procedures to produce disease-fighting cells that are personalized to a patient. For example, CAR-T (Chimeric Antigen Receptor T-cell) therapy is a cancer immunotherapy treatment that harnesses the power of a patient's immune system to combat cancer. CAR-T therapy and related clinical research involves meticulous orchestration of a series of steps that are frequently adapted as the process proceeds. A typical process begins with the extraction of the patient's T cells through apheresis, a procedure where blood is drawn and separated to isolate the immune cells. Subsequently, these T cells are transported to a specialized laboratory where they undergo genetic modification to express a Chimeric Antigen Receptor (CAR) that specifically targets cancer cells. The genetically modified CAR-T cells are then expanded and cultured to achieve a therapeutic dose. Following this, the patient undergoes a conditioning regimen to create an environment conducive to CAR-T cells. Finally, the modified cells are infused back into the patient, where they operate to destroy cancer cells expressing the targeted antigen. Following initial treatment, medical providers may continuously manage patient progress and monitor for potential side effects, such as cytokine release syndrome and neurotoxicity.

Other types of cell manufacturing processes may involve manufacturing personalized cells of other types such as natural killer (NK) cells, mesenchymal cells, dendritic cells, or other types of immune effector cells. These processes may similarly involve collection of cells (e.g., skin cells, cardiac cells, blood cells, etc.) through various collection techniques, shipping of cells, manufacturing of personalized cells, and infusion of cells into the patient.

Cell manufacturing processes involve close coordination between medical facilities or clinical researchers that manage patients and/or trial participants, manufacturing facilities that manufacture personalized cells, shipping/logistic services that manage transport, and other entities involved in the end-to-end process. Any delays or unexpected changes to the event schedule can have severe negative consequences. For example, patient schedule delays may cause manufacturing facilities to function sub-optimally, wasting manufacturing capacity that could have been used to help another sick patient. Delays and unexpected changes in the manufacturing steps can postpone treatment to the point of endangering patient lives. Furthermore, given the high cost of the cell manufacturing process, any errors or delays can be financially catastrophic for the various organizations involved.

### SUMMARY

A computer-implemented method manages a cell manufacturing process using a machine learning model to optimize event management. A cell manufacturing management platform obtains patient data for a patient and obtains an initial protocol for the cell manufacturing process for the patient. The cell manufacturing management platform applies a machine learning model to the patient data and the initial protocol to infer an initial planned sequence of events for the cell manufacturing process. The machine learning model is trained based on historical cell manufacturing processes and is trained to optimize an operational efficiency metric associated with the historical cell manufacturing processes. The cell manufacturing management platform facilitates tracking and updating of the planned sequence of events by iteratively performing a set of tracking and inference steps. These steps include obtaining tracking data for tracking progress of the cell manufacturing process, storing the tracking data to an event tracking log associated with the cell manufacturing process, re-applying the machine learning model to the patient data and the tracking data to update the planned sequence of events for the cell manufacturing process, and deriving one or more actions associated with the planned sequence of events. The cell manufacturing management platform then communicates, over a network, action data for facilitating performance of the one or more actions.

In an embodiment, communicating the action data comprises generating a user interface associated with the cell manufacturing process for the patient that includes a representation of the planned sequence of events, receiving, over a network, an access request from a client device to access the user interface including the representation of the planned sequence of events, and responsive to the access request, outputting the user interface to the client device.

In an embodiment, communicating the action data comprises generating a hard recommendation to halt the cell manufacturing process, and automatically disabling actions in the user interface associated with continuing the cell manufacturing process.

In an embodiment, communicating the action data comprises generating a soft recommendation to halt the cell manufacturing process, and communicating the soft recommendation to one or more client devices.

In an embodiment, communicating the action data comprises generating a notification relating to an upcoming event in the planned sequence of events, and communicating the notification to one or more client devices.

In an embodiment, communicating the action data comprises obtaining and storing an acknowledgement message from the one or more client devices responsive to the notification.

In an embodiment, communicating the action data comprises facilitating acquisition of a digital affirmation relating to the cell manufacturing process; and storing the digital affirmation.

In an embodiment, communicating the action data comprises assigning an action associated with an event to one or more parties, and communicating the assignment to a client device associated with the one or more parties.

In an embodiment, the machine learning model is trained according to a training process comprising obtaining, over a network, training data for training the machine learning model, the training data including patient data relating to patients that have participated in historical cell manufacturing processes and event data relating to historical events of the historical cell manufacturing processes, applying a machine learning algorithm to the training data to train the machine learning model based on the operational efficiency metric, and storing the machine learning model.

In further embodiments, a non-transitory computer-readable storage medium stores instructions executable by a processor for carrying out any of the processes described herein. In yet a further embodiment, a computer system includes one or more processors and a non-transitory computer-readable storage medium stores instructions executable by a processor for carrying out any of the processes described herein.

### BRIEF DESCRIPTION OF THE DRAWINGS

FIG. 1 is an example embodiment of a cell manufacturing process for cell therapy.

FIG. 2 is an example embodiment of a computing environment for a cell manufacturing management platform.

FIG. 3 is an example embodiment of a cell manufacturing management platform.

FIG. 4 is an example embodiment of interactions associated with a data collection module.

FIG. 5 is an example embodiment of interactions associated with an action module.

FIG. 6 is an example embodiment of a machine learning engine for a cell manufacturing management platform.

FIG. 7 is an example embodiment of a process for generating facilitating managing a cell manufacturing process using machine management platform.

FIG. 8 is a first example user interface associated with a cell manufacturing management platform.

FIG. 9 is a second example user interface associated with a cell manufacturing management platform.

FIG. 10 is a third example user interface associated with a cell manufacturing management platform.

FIG. 11 is a fourth example user interface associated with a cell manufacturing management platform.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

A cell manufacturing management platform facilitates management of a cell manufacturing process. The cell manufacturing management platform tracks events associated with a cell manufacturing process and coordinates between disparate entities involved in the process. The cell manufacturing management platform utilizes machine learning techniques to generate inferences associated with event scheduling in a manner that optimizes an efficiency metric and reduces likelihood of exceptions occurring. Machine learning models may furthermore be used to generate various alerts or other actions associated with the process. A user interface enables different participating entities to track progress of the process and upcoming events.

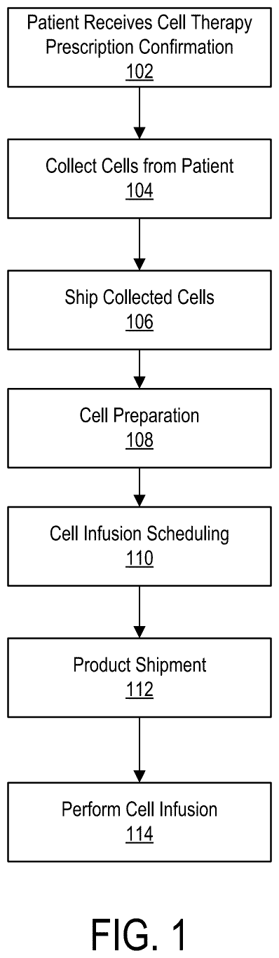

FIG. 1 illustrates a high-level example of a cell manufacturing process. The process begins with a patient receiving 102 a prescription for a cell-based therapy such as CAR-T. Alternatively, the cell-based therapy may relate to natural killer cells, mesenchymal cells, dendritic cells, or other immune effector cells. At a collection appointment, cells are collected 104 from the patient. For CAR-T, collection may be performed using apheresis, a medical technology in which blood is drawn and passed through an apheresis machine that separates T cells from the blood. For other types of cell therapy, different collection techniques may be performed which may involve collection of other types of cells such as skin cells, cardiac cells, or other cells. The obtained cells are then shipped 106 to a manufacturing facility. The manufacturing facility prepares 108 manufactured cells from the received cells. For example, in CAR-T, the manufacturing process involves production of T cells personalized to the patient. In other processes, the manufacturing process involves production of other types of immune effector cells. Next, an infusion appointment is scheduled 110 with the patient. The manufactured cell product is then shipped 112 to the medical facility for infusion. A cell infusion process may then be performed 114 to infuse the manufactured cells into the patient's bloodstream.

The very general process of FIG. 1 may be governed by a protocol comprising a set of rules or guidelines for controlling the process. The protocol may specify an order for performing at least the high-level steps. For example, the protocol may logically specify performing cell collection prior to shipping, receiving the cells at the manufacturer prior to initiating the manufacturing process, etc. The rules of the protocol may furthermore specify various conditions for beginning and/or completing each step of the above-described process. These conditions may relate to notifying various individuals about scheduled events, obtaining acknowledgements and/or consents, confirming that test results meet specifications, preparing lab equipment, confirming that medical equipment is operating properly, etc. For example, the protocol may specify that prior to collecting 104 cells, a set of sub-steps shall first be performed including: (1) confirming scheduling of an apheresis appointment; (2) ensuring that equipment is available and ready for use, (3) confirming availability of medical practitioners to perform the collection; (4) ensuring that downstream shipping and manufacturing steps can be performed within requisite time periods after collection, (5) ensuring that all consents are obtained from patients, medical providers, or other individuals, etc. As another example, prior to shipping 106 the collected cells, the protocol may require sub-steps such as (1) confirming that the manufacturing facility is ready and available to receive the shipment (2) ensuring that the shipping provider is available to perform the shipment; (3) ensuring that the collected cells are of sufficient number and quality for the manufacturing process, etc. The sequence of events defined by a protocol are not necessarily linear and may include rules specifying various branches, loops, or conditional steps. Furthermore, the protocol may specify halting the process under certain conditions.

The protocol may also include rules specifying certain thresholds or conditions that must be met at each step or sub-step. For example, the protocol may specify a required cell count and/or quality level of cells for the collection to be deemed compliant. Furthermore, the protocol may dictate that cells should be frozen using a control rate freezing process over a certain time period prior to shipping. In another example, the protocol may dictate that the cells must ship within a certain time period after freezing and may only be in transit for a limited time period.

An exception may occur when a rule of the protocol is not met. For example, a cell collection exception may occur when collected cells fail to meet the requisite cell count and/or quality, when a shipping delay occurs, when a patient misses an appointment, etc. Exceptions may add significant complications because not only must the exception be remedied, but it may trigger various delays that may cause further downstream exceptions if not adequately addressed. For example, when the cell count falls outside of specifications, a medical provider may reperform the cell collection, which may delay the process and dictate events such as scheduling a new patient appoint, notifying various parties, obtaining new patient consent, rescheduling shipping, etc. In other instances, rather than performing the recollection process immediately, a medical provider may first implement medical procedure to increase the patient's cell count. This adds additional steps to the process and may similarly result in rescheduling various downstream events to accommodate the delay. Similarly, unexpected delivery delays (e.g., due to weather) may result in collected cells not being delivered to the manufacturing facility in the requisite time period, which may trigger an additional step of testing the quality of cells at the manufacturer, performing a recollection, providing relevant notifications, establishing new acknowledgements or consents, etc. In other examples, exceptions may occur when the cells do not ship within the specified time period, when sensors in the freezer indicate that the temperature is outside of the specified range, when a patient misses an appointment, when manufacturer equipment fails, etc.

In some instances, a protocol may include rules for handling certain types of exceptions. However, the initial protocol generally does not account for all possible deviations from the initial event timeline. Thus, a cell manufacturing process frequently involves various decision making outside of the initial protocol. For example, an initial protocol may not specifically dictate how to handle a scheduling delay exception because scheduling may be subject to availability and consent of the various parties. Additionally, some decision points may be left to a medical provider (e.g., manner of increasing cell count in the event of an inadequate collection) and are not necessarily specified in the initial protocol. Based on these external factors, a protocol may adapt as the process progresses depending on the specific circumstances.

Thus, while FIG. 1 represents only a highly simplified cell collection process, a real-world process can be significantly more complicated based on the specific protocol and various exceptions that may occur while carrying out the protocol. Processes may start with an initial protocol outlining a planned sequence of events, but the planned event sequence may evolve as the process is carried out. In practice, a traditional cell manufacturing process may involve significant decision making at different steps based on case-specific factors such as the patient profile and medical history, manufacturer or provider-specific procedures, and the past history of events. Careful coordination is often needed between the patient, medical providers, manufacturers, shipping agents, or other entities involved in the process.

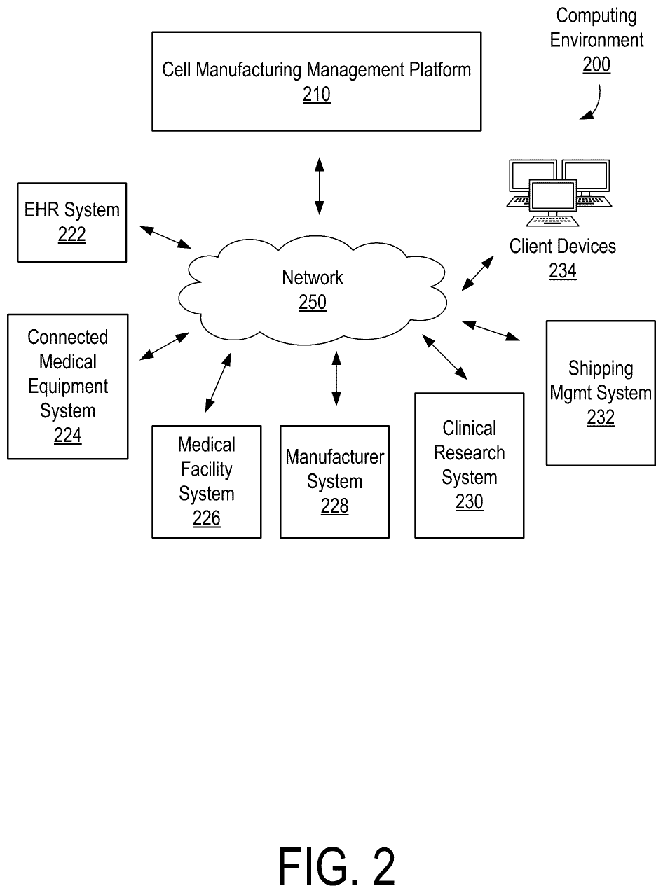

FIG. 2 illustrates an example embodiment of a computing environment 200 associated with automatically managing a cell manufacturing process. The computing environment 200 includes a cell manufacturing management platform 210 that interfaces with various systems over a network 250 such as, for example, an electronic healthcare records (EHR) system 222, connected medical equipment system 224, a medical facility platform system 226, a manufacturer system 228, a clinical research system 230, and a shipping management system 232. Different combinations of these systems may be involved with different cell manufacturing processes. For example, the clinical research system 230 may be utilized for cell manufacturing associated with a clinical research effort, while the medical facility system 226 may be involved for managing a cell therapy process for a patient being treated at a medical facility. The computing environment may also include multiple instances of various types of connected systems. For example, the cell manufacturing management platform 210 may interoperate with various medical facility systems operated by different medical facilities. Furthermore, in some scenarios, multiple systems may be integrated together. For example, a medical facility system 226 could include an integrated EHR system 222 and may also include its own connected medical equipment system 224 for managing medical equipment within the facility.

The cell manufacturing management platform 210 tracks and automates various management tasks associated with a cell manufacturing process in view of the complexities described above. The cell manufacturing management platform 210 may start with an initial protocol for a patient (or may automatically select between different preconfigured initial protocol) that is characterized by a sequence of planned events on an initial event timeline. The cell manufacturing management platform 210 tracks events as they occur, identifies exceptions, and intelligently manages updates to the planned event sequence (including types of events and timing of the events) based on the tracked events. The cell manufacturing management platform 210 may furthermore track operational data from medical equipment, which may further inform event scheduling. Management tasks facilitated by the cell manufacturing management platform 210 may include, for example, selecting or recommending an initial cell manufacturing protocol applicable to a patient, scheduling of events associated with the manufacturing process, tracking of event status, identifying event exceptions, determining protocol updates such as addition of events, removal of events, changing of events, reordering of events, or rescheduling of events, facilitating messages informative of tracked status and/or updates to the protocol, soliciting, obtaining, and tracking acknowledgements of receipt of the messages, obtaining affirmations associated with recommended actions, obtaining consent associated with events, etc. To facilitate these tasks, the cell manufacturing management platform 210 may interface with the various connected platforms and devices (e.g., EHR System 222, connected medical equipment 224, medical facility platform 226, manufacturer system 228, clinical research system 230, shipping management system 232, and client devices 234) to obtain data from these data sources and to output relevant updates. For example, the cell manufacturing management platform 210 may interoperate with connected platforms via an application programming interface (API) accessible over the network 250.

As events are tracked during the cell manufacturing process, the cell manufacturing management platform 210 may recommend and/or automatically enact updates to the future planned event sequence. Updates may include adding events, removing events, changing events, reordering events, and/or rescheduling events in response to tracked activities. For example, the cell manufacturing management platform 210 may determine, based on the tracked events and current future planned events, when rescheduling of future events is desirable (e.g., due to a delay, failed test, unavailability, or other condition). Updates could further include automatically halting the cell manufacturing process or generating recommendations to halt the process until an exception is remedied. The cell manufacturing management platform 210 may furthermore select between different potential timing of rescheduled events to optimize between various tradeoffs (e.g., time efficiency, likelihood of an exception occurring, etc.) In another example, when a shipping delay occurs, the cell manufacturing management platform 210 may intelligently determine (or recommend) whether to continue the process with the same collected cells (at risk of a quality check failing upon receipt), or immediately initiating scheduling of a new collection process. In further examples, the cell manufacturing management platform 210 may select between various mitigation strategies in response to an exception. For example, in response to cells failing to meet a quality check, the cell manufacturing management platform 210 may determine whether to schedule a new cell collection, to recommend medication for increasing cell count, or some other strategy. The cell manufacturing management platform 210 may furthermore intelligently facilitate rescheduling of downstream events to accommodate the selected strategy. In further examples, the cell manufacturing management platform 210 may further directly interact with medical equipment 224 to obtain data from these systems relevant to managing the cell manufacturing process, controlling timing of maintenance and/or calibration processes, monitoring operation, etc. In yet further examples, the cell manufacturing management platform 210 may intelligently select mechanisms and timing for informing various entities of updates, obtaining consent from different entities, or otherwise communicating with entities involved in the process in a manner that promotes high efficiency. The cell manufacturing management platform 210 may generate updates in a manner that may be patient specific. For example, a patient's general characteristics, health history, diagnosis, or other factors may lead to different updates than a differently situated patient.

In an example implementation, the cell manufacturing management platform 210 may utilize various machine learning techniques to intelligently facilitate management of the cell manufacturing process. In a training process, the cell manufacturing management platform 210 learns one or more machine learning model based on large sets of training data characterizing historical cell manufacturing processes. In this learning process, the cell manufacturing management platform 210 models how different event sequences (including relative timing of events) affect overall performance of the process given the initial protocol, history of tracked events, patient information, or other information. For example, when an exception occurs, the cell manufacturing management platform 210 may automatically generate a set of future events that are predicted to carry out the remaining protocol in the most effective manner. In an example embodiment, the machine learning model is trained to optimize an efficiency metric associated with the cell manufacturing process. The efficiency metric may characterize one or more parameters such as end-to-end process duration (e.g., from cell collection to infusion), number of exception events, number and/or duration of delays, number of rescheduling tasks relative to initial protocol, number of warnings triggered, patient outcomes (e.g., avoidance of chemotherapy), or other parameters (or combinations of parameters) associated with performance of the cell manufacturing process.

The cell manufacturing management platform 210 may also utilize machine learning techniques to assist decision making relating to individual events or sets of events. For example, machine learning models may be trained and applied to inform likelihoods of exceptions occurring and solutions for avoiding such exceptions. Various machine learning predictions can be used to generate various warning, recommendations, or other information relating to process to automatically enact actions or recommend actions to various individuals managing the process.

In further embodiments, the cell manufacturing management platform 210 may employ a combination of rule-based techniques and machine learning techniques to generate actions, recommendations, or other outputs for managing the cell manufacturing process. Examples of machine leaning techniques are described in further detail below.

The cell manufacturing management platform 210 may be implemented using on-site computing or storage systems, cloud computing or storage systems, or a combination thereof and may be implemented utilizing local or cloud-based servers, which may include physical or virtual machines, or a combination thereof. Cloud-based servers may include private cloud systems, public cloud systems, hybrid public/private cloud systems, or a combination thereof. Accordingly, the cell manufacturing management platform 210 may be local, remote, and/or distributed relative to the medical environments where procedures are performed and relative to the client devices 234 and other platforms (e.g., EHR system 222, connected medical equipment 224, medical facility platform 226, manufacturer system 228, clinical research system 230, and shipping management system 232). Furthermore, different portions of the cell manufacturing management platform 210 may execute on different remote servers and various system elements of the cell manufacturing management platform 210 may be communicatively coupled over a network 250.

The client devices 234 may include any computing devices for accessing data associated with the cell manufacturing management platform 210, inputting data to the cell manufacturing management platform 210, or otherwise interacting with the cell manufacturing management platform 210. Client devices 234 may similarly interact with one or more of the EHR system 222, connected medical equipment 224, medical facility platform 226, manufacturer system 228, clinical research system 230, and/or shipping management system 232. The client devices 234 may comprise, for example, a mobile phone, a tablet, a laptop or desktop computer, or other computing device. The client devices 234 may execute one or more applications including a user interface for viewing and/or editing information associated with the cell manufacturing management platform 210. For example, the application may comprise a web-based application accessible by a web browser or a locally installed application. The client devices 234 may include conventional computer hardware such as a display, input device (e.g., touch screen), memory, a processor, and a non-transitory computer-readable storage medium that stores instructions for execution by the processor in order to carry out functions described herein.

The various platforms (e.g., EHR system 222, connected medical equipment 224, medical facility platform 226, manufacturer system 228, clinical research system 230, and shipping management system 232) facilitate diverse services that may interact with the cell manufacturing management platform 210 in different ways. These platforms may similarly each be implemented using various on-site computing or storage systems, cloud computing or storage systems such as private cloud systems, public cloud systems, hybrid public/private cloud systems, or a combination thereof. The systems may utilize various databases, datasets, management logic, user interfaces, or other elements to facilitate the functions described herein.

The connected medical equipment system 224 may manage various medical equipment such as apheresis machines for collecting blood, refrigeration and/or freezers for storing collected cells, flow cytometers, robotic systems, imaging systems, surgical tools, various devices for obtaining more general patient physiological or biological signals such as pulse rate, blood pressure, body temperature, etc. These devices may generate various telemetry data that may be accessed by the cell manufacturing management platform 210.

The network 250 comprises communication pathways for communication between the cell manufacturing management platform 210, the EHR system 222, the connected medical equipment 224, the medical facility platform 226, the manufacturer system 228, the clinical research system 230, the shipping management system 232, and the client devices 234. The network 250 may include one or more local area networks and/or one or more wide area networks (including the Internet). The network 250 may also include one or more direct wired or wireless connections (e.g., Ethernet, WiFi, cellular protocols, WiFi direct, Bluetooth, Universal Serial Bus (USB), or other communication link).

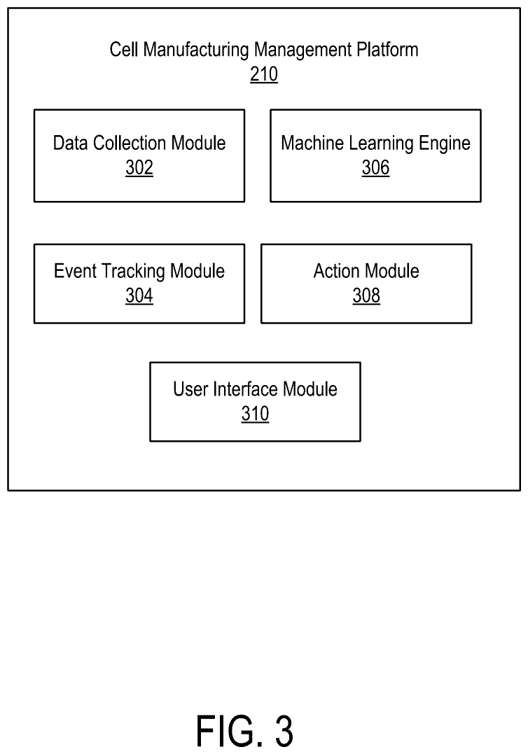

FIG. 3 is a block diagram of a cell manufacturing management platform 210. The cell manufacturing management platform 210 includes a data collection module 302, an event tracking module 304, a machine learning engine 306, an action module 308, and a user interface module 310. Alternative embodiments may include additional or different modules.

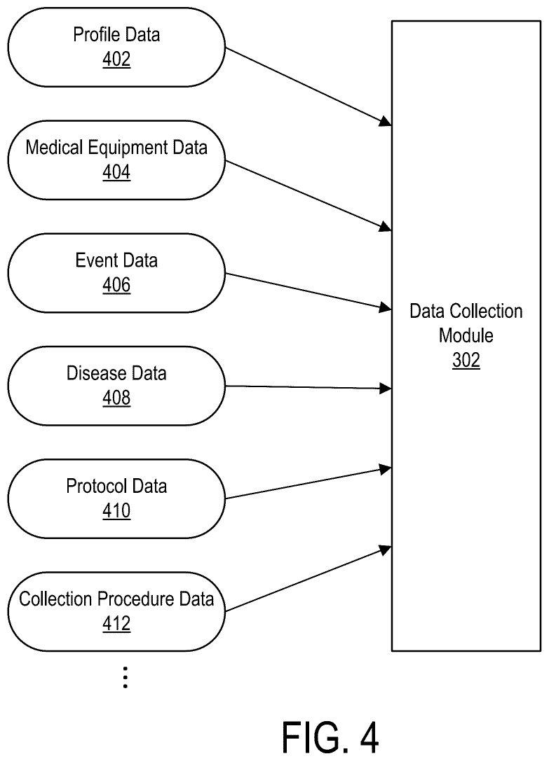

The data collection module 302 collects various data utilized by the cell manufacturing management platform 210. Types of collected data are shown in FIG. 4 and may include, for example, entity profile data 402, medical equipment data 404, event data 406, disease data 408, protocol data 410, collection procedure data 412, and/or other data types.

The profile data 402 may include characteristics of various entities such as patients, clinical trial participants, medical providers, medical facilities, clinical trial managers, manufacturers, shipping agents, logistics managers, etc. For patients, profile data 402 may include information such as age, smoking habits, drinking habits, fitness metrics, vitals, lab data, concomitant medications, previous treatment or medication history, genomic characteristics, human leukocyte antigen (HLA) typing, infectious disease markers (IDMs), disease diagnosis data, characteristics/subclassifications of diagnoses, cell pathology and characteristics data, protein electrophoresis data, cell collection characteristics and attributes pre-procedure, mobilization details, patient/donor education details, or other information relating to health history, medical conditions, lab results, biometric data, procedure performed, prescriptions, post-procedural outcomes. Patient profile data may be obtained from the EHR system 222 in some embodiments.

Profile data 402 associated with medical providers and clinical trial managers may include, for example, information about experience, expertise, procedures performed, etc. Profile data 402 associated with medical facilities may include staffing information, available expertise and experience, location information, time zone, available equipment, etc. Profile data 402 associated with manufacturers may include information about manufacturing protocols, cost information, capabilities, historical performance, machine availability, etc. Profile data 402 associated with shipping agents and logistics managers may include information about capabilities, availability, cost information, historical performance, etc.

Medical equipment data 404 may include information about medical equipment associated with cell manufacturing processes such as apheresis systems, flow cytometers, refrigeration and/or freezer systems, robotic systems, imaging systems, surgical tools, etc. or other equipment discussed herein that may be obtained from the connected medical equipment system 224. The medical equipment data 404 may include telemetry data collected from medical equipment as it relates to a cell manufacturing process. For example, the medical equipment data 404 may include temperature readings from refrigerators or freezers used to store cells, apheresis data monitored by apheresis machines, etc. Medical equipment data 404 may furthermore include data relating to machine calibration, maintenance, performance, or other characteristics that may affect operations.

Event data 406 may include time-based data associated with historical, ongoing, and/or future cell manufacturing processes. Each event may include a timestamp indicating when the event occurred or is scheduled to occur and event data associated with the event. Examples of events may include scheduling events (e.g., scheduling of patient appointment, scheduling of shipping, scheduling of a manufacturing process, etc.), testing events (e.g., testing of cells, testing of medical equipment, patient testing, etc.), notification events (e.g., sending a notification to a patient, medical provider, manager, shipping agent, etc.), affirmation or acknowledgement events (e.g., obtaining acknowledge of receipt of information and/or expressly obtaining consent for an action), assignment events (e.g., assigning an action to a provider, patient, manager, medical equipment, etc.) action events (e.g., performing a medical procedure such as cell collection or infusion, performing a test such as testing cell quality testing cell counts or testing medical equipment, initiating a shipment, performing a manufacturing process, performing cell infusion, etc.), or other types of time-based events associated with the cell manufacturing process.

Event data 406 may relate to different stages of a cell manufacturing process. For example, preprocedural event data may characterize information such as a patient identifier, gender, weight, fluid balance information, apheresis machine information, and various procedure details. Post collection data may relate to storage time of collected cells, cryopreservation time, storage locations and associated data, shipping methods and times, cell counts, cell viability and quantity, etc.

Disease data 408 may include information describing various diseases. For example, disease data may include description of disease symptoms, diagnosis techniques, prognosis, treatment methods, statistical information, clinical research results, or other data. As it pertains to a particular patient, disease data 408 may include information relating to a patient's diagnosis with a disease, prognosis, and current or historical treatments.

Protocol data 410 may include a set of steps (which may include respective pre-steps, sub-steps, or post-steps) and rules for managing events in a cell manufacturing process. Protocol rules may control when a step is considered complete, conditions for advancing to a subsequent step, conditions for identifying an exception, conditions for selecting between different possible branches, conditions for repeating steps, conditions for skipping steps, conditions for reordering steps, or conditions for otherwise updating the protocol. Protocols may be dependent on various factors including the identity of the patient, medical provider, and manufacturer, the type of treatment being provided, the patient's medical state, availability of different entities involved, etc. Protocols may change throughout a process and the protocol data may therefore include updates to the protocol that may occur during a process. Protocol updates may include rescheduling of events, changing of events, adding events, subtracting events, or reordering events.

Collection procedure data 412 may include information about cell collection or collection of other biological samples from patients. This data may describe various collection methods and/or provide statistical data relating to collection methods historically used.

Referring back to FIG. 3, The data collection module 302 may aggregate data from various input data sources. For example, the data collection module 302 may perform various pre-processing to normalize data to a standardized format used by the cell manufacturing management platform 210, filter data, index data, combine data, sort data, or otherwise process data for use by the cell manufacturing management platform 210.

The data collection module 302 may be electronically coupled to one or more external servers, databases, or other data sources that supply the data. For example, data may be sourced from any of the EHR system 222, connected medical equipment system 224, medical facility system 226, manufacturer system 228, clinical research system 230, shipping management system 232, directly from client devices 234, or from other servers not expressly shown in FIG. 2 (e.g., public data sources such as the internet, or various private databases).

The data collection module 302 may furthermore provide an application programming interface (API) to enable it to seamlessly collect data from the various information sources shown in FIG. 2. Alternatively, or in addition, the data collection module 302 may operate according to one or more APIs managed by the data sources (e.g., an API associated with a specific EHR system 222). The data collection module 302 may furthermore provide interfaces for direct data entry via the client devices 234 through web forms, applications, or other interfaces.

In an embodiment, the data collection module 302 may collect and manage data in a manner consistent with various compliance and privacy policies. For example, the data collection module 302 may enable removal or redaction of portions of received data to preserve privacy of a patient dependent on configured privacy policies, intended use of the data, or other parameters.

The event tracking module 304 tracks events associated with a cell manufacturing process based on information received through the data collection module 302. For example, for a given process for a patient, the event tracking module 304 may maintain an event log of tracked events with each event defined by a timestamp and event data. The event tracking module 304 may update the event log each time new relevant data is received. The event tracking module 304 may furthermore perform various aggregations or other processing to maintain the event log in a form suitable for use by other modules of the cell manufacturing management platform 210. Event logs may be organized on a process-by-process basis with each cell manufacturing process characterized by its own event log.

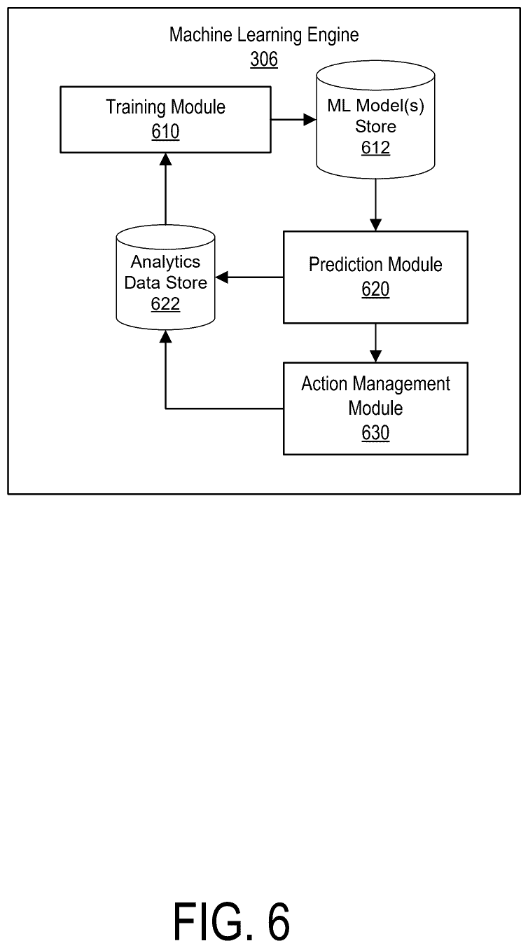

The machine learning engine 306 performs various machine learning functions to train and apply a machine learning that can generate updates to a cell manufacturing process. The machine learning model may be trained based on historical cell manufacturing process data (e.g., any of the types of data collected by the data collection module 302 described above). In an embodiment, the machine learning engine 306 is trained to infer updates to a planned cell manufacturing event sequence based on tracked events, patient characteristics, or other factors in a manner that optimizes an efficiency metric associated with the cell manufacturing process. An example embodiment of a machine learning engine 306 is described in further detail below with respect to FIG. 6.

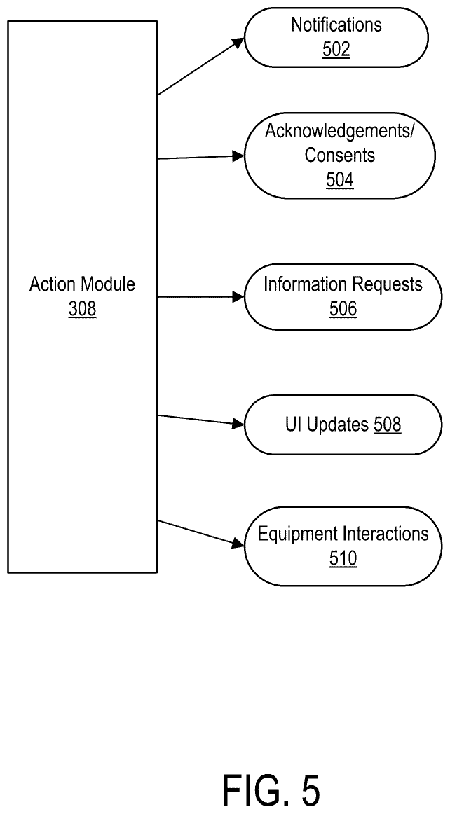

The action module 308 facilitates various actions to carry out aspects of a cell manufacturing process. For example, as shown in FIG. 5, the action module 308 may facilitate various actions such as notifications 502, collection of acknowledgements and/or consents (e.g., signatures) 504, solicitation of information 506 from various entities (e.g., confirming availability for scheduling, requesting test results, etc.), performing updates 508 to a user interface, or facilitating various equipment interactions 510 (e.g., running a freezer calibration, generating a shipping label, etc.). The action module 308 may communicate with various systems and/or clients 234 connected to the network 250 using various communication protocols such as text messaging, email, push notifications, robocalling, chatbots, or other communication methods. The action module 308 may store communication preferences associated with different entities and facilitate communication with each entity based on their respective preferences. The action module 308 may furthermore present updates in a user interface accessible via a web site or computer application to present notifications or solicit consents or other inputs from different entities. Furthermore, the action module 308 may facilitate communications with the various systems shown in FIG. 2 (e.g., EHR system 222, connected medical equipment 224, medical facility platform 226, manufacturer system 228, clinical research system 230, and shipping management system 232) via one or more APIs.

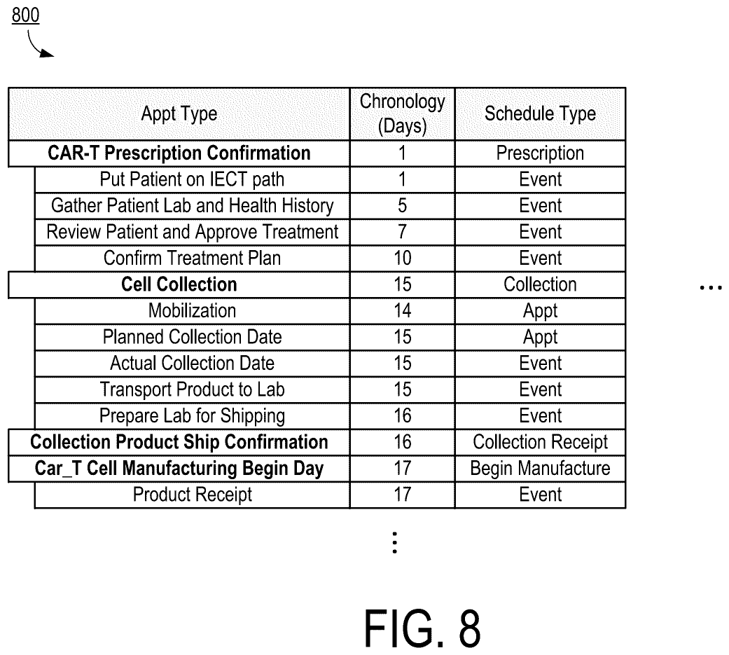

Referring back to FIG. 3, The user interface module 310 facilitates presentation of one or more user interfaces associated with the cell manufacturing management platform 210. The user interface module 310 may comprise a webpage accessible via a web browser, a mobile application interface screen, desktop application interface screen, a voice assistant, chatbot, or other interface that enables users to access information of the cell manufacturing management platform 210 and/or input information to the cell manufacturing management platform 210. For example, a user interface module 310 may present a user interface associated with an ongoing cell manufacturing process that presents a timeline of completed and/or scheduled events associated with the process. As the process progresses, the user interface may automatically update the presentation to present changes such as rescheduling of events, adding or removing events, repeating events, etc. The user interface module 310 may also facilitate presentation of various alerts, which may be generated based on a set of alert rules and/or based on predictions from the machine learning engine 306. Examples of user interfaces and alerts are described below with respect to FIGS. 8-11.

FIG. 6 is a block diagram of a machine learning engine 306 associated with a cell manufacturing management platform 210. The machine learning engine 306 comprises a training module 610, a machine learning (ML) model store 612, a prediction module 620, an action management module 630, and an analytics data store 622.

The training module 610 executes one or more machine learning algorithms to learn parameters for one or more machine learning models for generating updates to a planned event sequence associated with a cell manufacturing process. The training module 610 obtains historical data associated with cell manufacturing processes and derives feature vectors from the training data. Here, each feature vector may correspond to a single historical cell manufacturing process. The feature vector may capture information about the patient, medical providers, manufacturer, shipping agents, facilities, medical equipment, and/or other entities involved in the process. The feature vector may furthermore represent an initial protocol for the cell manufacturing process and the time-based sequence of events that occurred during the manufacturing process. In some embodiments, the feature vector may directly characterize changes to the protocol (e.g., in response to exceptions) during the historical process. For example, instead of only characterizing the actual observed event sequence, the feature vector could characterize the planned sequence of events each time it changes throughout a process. The feature vector associated with a historical cell manufacturing process may furthermore characterize various analytics associated with the cell manufacturing process. The analytics may characterize efficiency or other performance metrics such as the time duration of the process, number of exceptions, number or duration of delays, or other characteristics or combinations thereof.

In another embodiment, multiple feature vectors may be generated for each historical cell manufacturing process, which each correspond to a different state of the process at different time instances. For example, a feature vector associated with a first state of a process may characterize a cell manufacturing process at a particular time instance by representing the set of past events that have occurred prior to the time instance and the set of planned future events. Another feature vector may represent another state of the same process at a later time instance by similar characterizing the set of past events that occurred prior to the later time instance and the current set of planned future events.

Based on the set of feature vectors for the historical processes in the training dataset, the training module 610 learns parameters (e.g., weights) for one or more machine learning models that map between the feature vectors and their respective performance metrics. In this process, the training module 610 may learn how different characteristics of actual or planned event sequences (together with information about patient characteristics, facilities, medical equipment, and other collected data) correlate with different efficiency outcomes. For example, the training module 610 may learn that requesting patient consent one week before the appointment is significantly more efficient than waiting until the day before. In another example, the training module 610 may learn that patients with certain medical conditions are more likely to fail a cell quality test, and that for such patients, it is more efficient to budget extra time for recollection from the outset than wait for the results and reschedule. In another example, the training module 610 could learn that exceptions are likely to occur when a specific small manufacturer receives shipments on Sunday, and that efficiencies can be gained by scheduling delivery for that manufacturer on a different day.

While the above examples are simplified representations of the outcomes of the machine learning process, the training module 610 in practice does not necessarily learn such correlations in isolation, but instead learns statistical correlations in the training set that may be intertwined in complex ways. Thus, the learned correlations may relate to multiple interrelated factors that collectively affect process efficiency.

In further embodiments, the training module 610 may employ train multiple machine learning models that make predictions about different specific aspects of a cell manufacturing process. For example, the training module 610 might train a specific machine learning model to predicts likelihoods of a patient needing a hospital bed on different days. In other example, the training module 610 could train a machine learning model to predict a likelihood of a cell quality test for a patient being within specifications. In another example, the training module 610 may train a machine learning model to predict a likelihood of a shipment arriving within a specified time window. These types of models could be used in combination with other models and/or rule-based decision models to facilitate management of the cell manufacturing process.

The training module 610 may employ various machine learning techniques such as, for example, neural networks (such as convolutional neural network (CNN), artificial neural network (ANN), residual neural network (ResNet), or recurrent neural network (RNN)), regression-based models, generative models, Large Language Models (LLMs) to analyze text-based content, or other type of machine-learned model capable of achieving the functions described herein.

The prediction module 620 predicts performance associated with a currently planned event sequence for a cell manufacturing process and/or possible updates to the event sequence. Updates may include updates to the types and ordering of the planned events and/or to the planned timing of such events. To generate predictions, the prediction module 620 may obtain various data from the data collection module 302 relating to a planned or ongoing cell manufacturing process and generate one or more feature vectors characterizing the process. The form of the feature vector may be similar to the features vectors generated in the training process described above. For example, feature vector may characterize profile information about the patient, facilities, medical providers, manufacturer, etc., the initial and/or current planned protocol for the process, and the event log of tracked events that have occurred during the process.

As new events are logged (whether events are successfully completed or whether an exception occurs), the prediction module 620 applies the one or more machine learning models to evaluate predicted performance under the current planned sequence, and may evaluate performance under one or more changes to the current planned sequence.

The prediction module 620 may furthermore apply aspect-specific models trained to predict likelihoods of certain occurrences. For example, the prediction module 620 may apply a machine learning model trained to predict likelihood of a patient needing a hospital bed on one or more days, a likelihood of a cell quality test for a patient being within specification, a likelihood of a shipment arriving within a specified time window, etc.

The prediction module 620 may apply an inference algorithm employing machine learning techniques similar to those used by the training module 610 described above.

The action management module 630 obtains the predictions from the prediction module 620 and determines specific actions based on the predictions to be carried out by the action module 308 described above. For example, the action management module 630 may compare predicted efficiency metrics associated with different possible updates to the event sequence and select the update most likely to yield best performance. The action management module 630 furthermore may coordinate updates to multiple interconnected future events. For example, when a single event is delayed, the action management module 630 may generate updated events for multiple downstream events to reduce the likelihood of downstream exceptions. Additionally, the action management module 630 may identify when additional acknowledgements or consents are needed based on updates to other events, and infer the optimal timing of actions for obtaining such acknowledgements or consents.

In further embodiments, the action management module 630 may employ a combination of machine learning predictions and rule-based techniques to generate the updated sequence of events for a cell manufacturing process. For example, the action management module 630 may employ a rule-based approach to schedule a hospital bed for a patient whenever a cell infusion appointment is made and may apply a prediction from the prediction module 620 to predict the most likely day (or set of days) that the patient will be ready for the procedure. The action management module 630 could then apply various rules to determine mechanisms for facilitating the reservation, which may be carried out by the action module described above.

The analytics data store 622 stores various analytics associated with predictions and actions taken from those predictions. This information may then be used as additional training data that may be applied by the training module 610 to update the machine learning model.

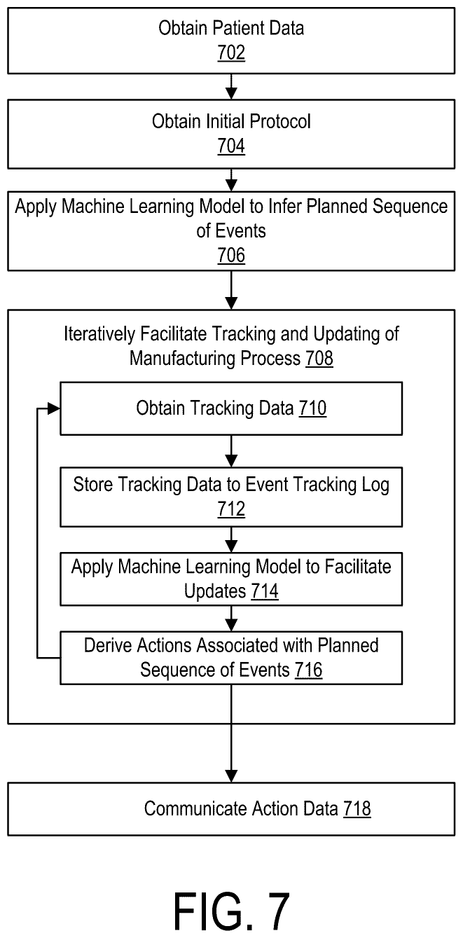

FIG. 7 illustrates an example embodiment of a process for managing a cell manufacturing process by a cell manufacturing management platform. The cell manufacturing management platform 210 obtains 702 patient data for a patient and obtains 704 an initial protocol for the cell manufacturing process associated with the patient. The cell manufacturing management platform 210 applies 706 a machine learning model to the patient data and the initial protocol to infer a sequence of events for the cell manufacturing process. The machine learning model may be trained based on historical cell manufacturing data for patients that have participated in cell therapy processes or clinical trials. The machine learning model may be trained to optimize an efficiency metric associated with the cell manufacturing process. The cell manufacturing management platform 210 facilitates 708 tracking and updating of the cell manufacturing process in an iterative manner. Over multiple iterations, the cell manufacturing management platform 210 tracks 710 events associated with the cell manufacturing process. The tracked events may include temporal information describing timing of the tracked events and event data describing characteristics of the events. The tracked events are stored 712 to an event tracking log associated with the cell manufacturing process. The cell manufacturing management platform 210 re-applies 714 the machine learning model using the event tracking log to update the planned sequence of events for the cell manufacturing process. The cell manufacturing management platform 210 further derives 716 actions associated with the planned sequence of events. Steps 710-716 may be performed iteratively as new events are tracked such that the set of planned future events are periodically re-optimized.

The cell manufacturing management platform furthermore communicates 718 action data associated with the planned sequence of events. For example, notification events may be generated to notify one or more entities of a status of the process, a scheduled appointment, a test result, or other information. Other types of actions may include obtaining acknowledgements, obtaining consents, scheduling an activity, conducting a test, etc. In further embodiments, an action may include a hard or soft recommendation to halt the cell manufacturing process, which may put the process on hold until an exception is remedied.

In an embodiment, communicating 718 the action data may include generating a user interface that includes a representation of the planned sequence of events. In response to an access request from a client device, the cell manufacturing management platform 210 may output the interface. In further embodiments, communications may be implemented via email, text message, via a chatbot, or other form of digital communication. Communications may be transmitted to different entities and actions associated with different events may be sent to different entities. For example, events associated with patient scheduling may be sent to a medical provider but are not necessarily sent to a manufacturer.

FIG. 8 illustrates a first example user interface 800 associated with the cell manufacturing management platform 210. This interface 800 shows an example subset of events associated with a CAR-T process, current planned chronology for the events, and scheduling type. The events may be organized as main events (shown in bold) and sub-events that relate to the main event. The chronology represents the current planned time for the event (e.g., in days) measured from the start of the process. The interface 800 may dynamically update as the planned schedule changes due to delays, missed appointments, rescheduling, or other exceptions. As explained above, a change in timing of one event may cause updates to downstream scheduled events in a manner that model predicts will optimize the overall process.

The event timeline in FIG. 8 is just one example and the specific event schedule may vary between different protocols or based on other factors such as patient characteristics, manufacturer procedures, medical practitioner preferences, etc.

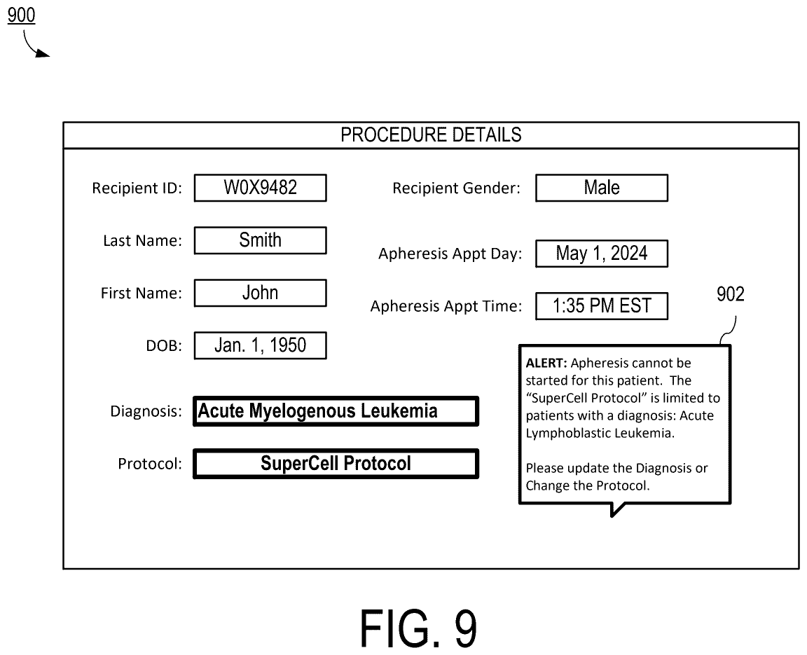

FIG. 9 illustrates a second example user interface 900 associated with the cell manufacturing management platform 210. In this example, the interface 900 displays patient data associated with a cell therapy process for a patient. The interface 900 also shows alert 902 that may be automatically generated by the cell manufacturing management platform 210. In this example, the alert indicates that there is a detected mismatch between the assigned protocol for the patient and the diagnosis. The alert may be based on inferences from the machine learning model and/or rule-based model. For example, in a rule-based approach, a set of rules may specify which protocols can be assigned for different diagnoses. In a machine learning approach, a rule is not necessarily specifically designated and the machine learning model. Instead, the alert and recommendation may be based on learned correlations in the training data (e.g., that exceptions are likely to occur in this scenario).

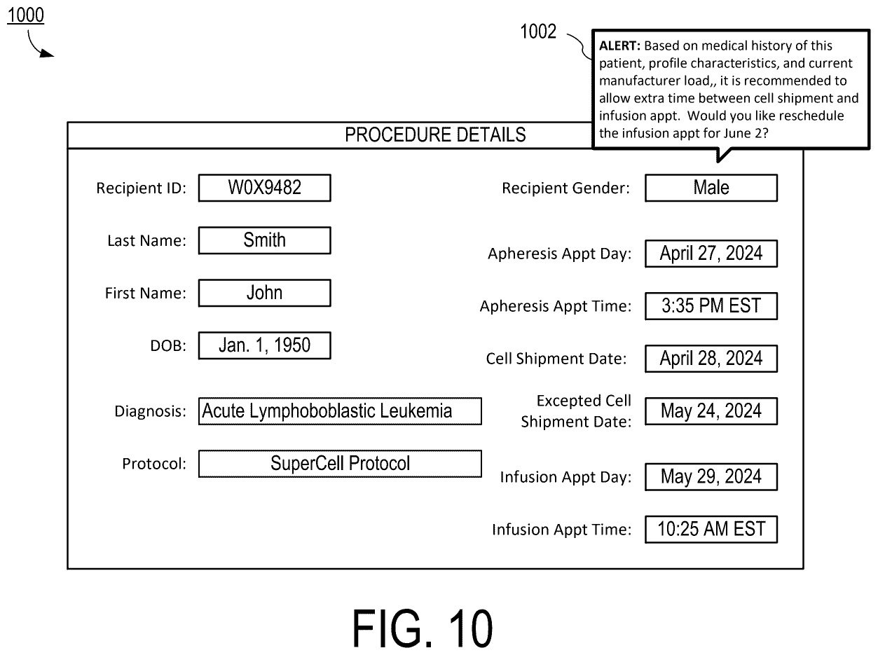

FIG. 10 illustrates a third example user interface 1000 associated with the cell manufacturing management platform 210. In this example, the interface 1000 displays patient data and some patient-specific scheduling details associated with a cell therapy process. An alert 1002 includes a recommendation to consider rescheduling an infusion appointment for a patient. The alert 1002 may be generated based on the machine learning model predicting that maintaining the current schedule is likely to result in scheduling exceptions and accordingly recommend a different schedule that optimizes efficiency of the process. Alternatively, the alert 1002 may be generated based on one or more rule-based techniques.

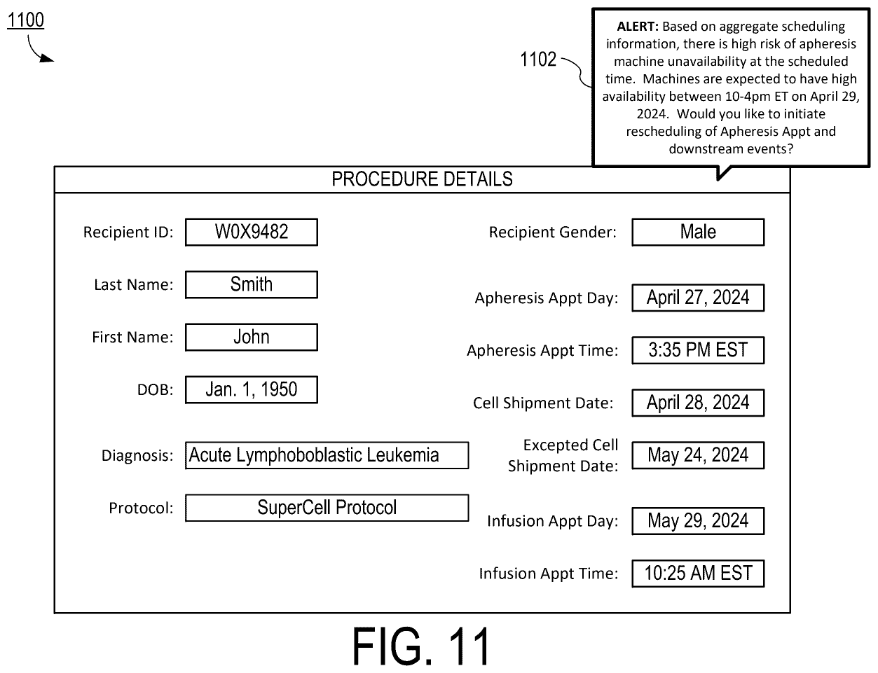

FIG. 11 illustrates a fourth example user interface 1100 associated with the cell manufacturing management platform 210. In this example, the interface 1100 displays patient data and some patient-specific scheduling details associated with a cell therapy process. An alert 1102 includes a recommendation to consider rescheduling an apheresis appointment for a patient in view of anticipated machine availability. If an administrator selects to reschedule, downstream scheduled events may be automatically rescheduled in a coordinated manner based on application of the machine learning model.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may include a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may include architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

What is claimed is:

1. A method for managing a cell manufacturing process using machine learning models to optimize event management, the method comprising:
obtaining patient data for a patient;
obtaining an initial protocol for the cell manufacturing process for the patient;
applying a machine learning model to the patient data and the initial protocol to infer an initial planned sequence of events for the cell manufacturing process, wherein the machine learning model is trained based on historical cell manufacturing processes, and the machine learning model is trained to optimize an operational efficiency metric associated with the historical cell manufacturing processes;
facilitating tracking and updating of the planned sequence of events by iteratively performing steps including:
obtaining tracking data for tracking progress of the cell manufacturing process;
storing the tracking data to an event tracking log associated with the cell manufacturing process;
re-applying the machine learning model to the patient data and the tracking data to update the planned sequence of events for the cell manufacturing process;
deriving one or more actions associated with the planned sequence of events; and
communicating, over a network, action data for facilitating performance of the one or more actions.

2. The method of claim 1, wherein communicating the action data comprises:
generating a user interface associated with the cell manufacturing process for the patient that includes a representation of the planned sequence of events;
receiving, over a network, an access request from a client device to access the user interface including the representation of the planned sequence of events; and
responsive to the access request, outputting the user interface to the client device.

3. The method of claim 1, wherein communicating the action data comprises:
generating a hard recommendation to halt the cell manufacturing process; and
automatically disabling actions in a user interface associated with continuing the cell manufacturing process.

4. The method of claim 1, wherein communicating the action data comprises:
generating a soft recommendation to halt the cell manufacturing process; and
communicating the soft recommendation to one or more client devices.

5. The method of claim 1, wherein communicating the action data comprises:
generating a notification relating to an upcoming event in the planned sequence of events; and
communicating the notification to one or more client devices.

6. The method of claim 5, wherein communicating the action data comprises:
obtaining and storing an acknowledgement message from the one or more client devices responsive to the notification.

7. The method of claim 1, wherein communicating the action data comprises:
facilitating acquisition of a digital affirmation relating to the cell manufacturing process; and
storing the digital affirmation.

8. The method of claim 1, wherein communicating the action data comprises:
assigning an action associated with an event to one or more parties; and
communicating the assignment to a client device associated with the one or more parties.

9. The method of claim 1, wherein the machine learning model is trained according to a training process comprising:
obtaining, over a network, training data for training the machine learning model, the training data including patient data relating to patients that have participated in historical cell manufacturing processes and event data relating to historical events of the historical cell manufacturing processes;
applying a machine learning algorithm to the training data to train the machine learning model based on the operational efficiency metric; and
storing the machine learning model.

10. A non-transitory computer-readable storage medium stores instructions for managing a cell manufacturing process using one or more machine learning models to optimize event management, the instructions for causing one or more processors to perform steps including:
obtaining patient data for a patient;
obtaining an initial protocol for the cell manufacturing process for the patient;
applying a machine learning model to the patient data and the initial protocol to infer an initial planned sequence of events for the cell manufacturing process, wherein the machine learning model is trained based on historical cell manufacturing processes, and the machine learning model is trained to optimize an operational efficiency metric associated with the historical cell manufacturing processes;
facilitating tracking and updating of the planned sequence of events by iteratively performing steps including:
obtaining tracking data for tracking progress of the cell manufacturing process;
storing the tracking data to an event tracking log associated with the cell manufacturing process;
re-applying the machine learning model to the patient data and the tracking data to update the planned sequence of events for the cell manufacturing process;
deriving one or more actions associated with the planned sequence of events; and
communicating, over a network, action data for facilitating performance of the one or more actions.

11. The non-transitory computer-readable storage medium of claim 10, wherein communicating the action data comprises:
generating a user interface associated with the cell manufacturing process for the patient that includes a representation of the planned sequence of events;
receiving, over a network, an access request from a client device to access the user interface including the representation of the planned sequence of events; and
responsive to the access request, outputting the user interface to the client device.

12. The non-transitory computer-readable storage medium of claim 10, wherein communicating the action data comprises:
generating a hard recommendation to halt the cell manufacturing process; and
automatically disabling actions in a user interface associated with continuing the cell manufacturing process.

13. The non-transitory computer-readable storage medium of claim 10, wherein communicating the action data comprises:
generating a soft recommendation to halt the cell manufacturing process; and
communicating the soft recommendation to one or more client devices.

14. The non-transitory computer-readable storage medium of claim 10, wherein communicating the action data comprises:
generating a notification relating to an upcoming event in the planned sequence of events; and
communicating the notification to one or more client devices.

15. The non-transitory computer-readable storage medium of claim 14, wherein communicating the action data comprises:
obtaining and storing an acknowledgement message from the one or more client devices responsive to the notification.

16. The non-transitory computer-readable storage medium of claim 10, wherein communicating the action data comprises:
facilitating acquisition of a digital affirmation relating to the cell manufacturing process; and
storing the digital affirmation.

17. The non-transitory computer-readable storage medium of claim 10, wherein communicating the action data comprises:
assigning an action associated with an event to one or more parties; and
communicating the assignment to a client device associated with the one or more parties.

18. The non-transitory computer-readable storage medium of claim 10, wherein the machine learning model is trained according to a training process comprising:
obtaining, over a network, training data for training the machine learning model, the training data including patient data relating to patients that have participated in historical cell manufacturing processes and event data relating to historical events of the historical cell manufacturing processes;
applying a machine learning algorithm to the training data to train the machine learning model based on the operational efficiency metric; and
storing the machine learning model.

19. A computer system comprising:
one or more processors; and
a non-transitory computer-readable storage medium stores instructions for managing a cell manufacturing process using one or more machine learning models to optimize event management, the instructions for causing the one or more processors to perform steps including:
obtaining patient data for a patient;
obtaining an initial protocol for the cell manufacturing process for the patient;
applying a machine learning model to the patient data and the initial protocol to infer an initial planned sequence of events for the cell manufacturing process, wherein the machine learning model is trained based on historical cell manufacturing processes, and the machine learning model is trained to optimize an operational efficiency metric associated with the historical cell manufacturing processes;
facilitating tracking and updating of the planned sequence of events by iteratively performing steps including:
obtaining tracking data for tracking progress of the cell manufacturing process;
storing the tracking data to an event tracking log associated with the cell manufacturing process;
re-applying the machine learning model to the patient data and the tracking data to update the planned sequence of events for the cell manufacturing process;
deriving one or more actions associated with the planned sequence of events; and
communicating, over a network, action data for facilitating performance of the one or more actions.

20. The computer system of claim 19, wherein communicating the action data comprises:
generating a user interface associated with the cell manufacturing process for the patient that includes a representation of the planned sequence of events;
receiving, over a network, an access request from a client device to access the user interface including the representation of the planned sequence of events; and
responsive to the access request, outputting the user interface to the client device.
